(19) United States
(12) Patent Application Publication
(10) Pub. No.: US 2022/0084383 A1
(43) Pub. Date: Mar. 17, 2022

(54) SYSTEM AND METHOD FOR MONITORING AN INDIVIDUAL USING LIDAR

(71) Applicant: Curbell Medical Products, Inc., Orchard Park, NY (US)

(72) Inventor: Thomas P. Kennedy, Lake View, NY (US)

(21) Appl. No.: 17/158,285

(22) Filed: Jan. 26, 2021

Related U.S. Application Data
(60) Provisional application No. 63/077,850, filed on Sep. 14, 2020.

(51) Int. Cl. G08B 21/02; G01S 7/481; G16H 40/40; G01S 17/89

(52) U.S. Cl. CPC G08B 21/02; G01S 17/89; G16H 40/40; G01S 7/4817

(57) ABSTRACT

A system for monitoring an individual includes a processor and a LIDAR sensor in electronic communication with the processor. The processor is configured to receive a set of spatial data from the LIDAR sensor, calculate a first location of the individual relative to a support object based on the set of spatial data, and determine if the first location is at an alert location relative to the support object. In another aspect, a method for monitoring an individual includes receiving a first set of spatial data from a LIDAR sensor, calculating a first location of the individual relative to a support object based on the first set of spatial data, and determining if the first location is at an alert location relative to the support object.

### CROSS-REFERENCE TO RELATED APPLICATIONS

This application claims priority to U.S. Provisional Application No. 63/077,850, filed on Sep. 14, 2020, now pending, the disclosure of which is incorporated herein by reference.

### FIELD OF THE DISCLOSURE

The present disclosure relates to monitoring the location of individuals for fall management and other purposes.

### BACKGROUND OF THE DISCLOSURE

Fall management of individuals in health care settings is an area of significant interest for healthcare providers. Individuals with limited mobility may have difficulty exiting a bed, a chair, or another support object without assistance. Such individuals are at high risk of a fall, which may exacerbate an existing condition or cause new injuries. An existing technology for monitoring individuals includes the use of a sensor pad placed under the person being monitored. This technology relies on the patient unloading the sensor pad (i.e., removing their weight from the sensor pad) in order to generate a signal to the caregiver.

Other existing technologies include outfitting the patient with motion, pressure, or other sensing devices, which often proves to be difficult to deploy and potentially unsanitary if the sensing devices are not easy to clean or disposable. Still other existing systems utilize video monitoring techniques, which raise concerns regarding the privacy of the individuals being monitored and are often costly to deploy and maintain.

There continues to be a need for a monitoring solution that is unobtrusive and sanitary, without creating privacy concerns.

### BRIEF SUMMARY OF THE DISCLOSURE

The present disclosure provides a system for monitoring an individual. The system includes a processor and a LIDAR sensor in electronic communication with the processor. The processor is configured to receive a set of spatial data from the LIDAR sensor; calculate a first location of the individual relative to a support object based on the set of spatial data; and determine if the first location is at an alert location relative to the support object. The processor may be configured to calculate the first location of the individual relative to the support object by distinguishing spatial data of the individual from spatial data of the support object, and calculating a center of mass of the individual based on the spatial data of the individual.

The processor may be further configured to determine an alert position of the individual relative to the support object by calculating a location of at least one edge of the support object. For example, the alert location may be determined to be where the center of mass of the individual is beyond the edge of the support object. In another example, the alert location may be determined to be where the center of mass of the individual is within a predetermined distance of the edge of the support object. The processor may be further configured to send an alert signal when the first location is determined to be at the alert location.

In some embodiments, the processor may be further configured to receive a second set of spatial data from the LIDAR sensor; calculate a second location of the individual relative to the support object based on the second set of spatial data; and determine if the second location is at an alert location relative to the support object. For example, the processor may be configured to determine if the change from the first location to the second location is indicative of movement to an alert location. The processor may be configured to calculate a probability that the individual will move to an alert location based on the first location and the second location. The processor may be configured to determine a direction of movement of the individual. The processor may be configured to determine a velocity of the individual. In some embodiments, the processor may be configured to determine if the individual has moved from a recumbent position to a sitting position based on the first location and the second location.

In some embodiments, the processor may be configured to receive one or more additional sets of spatial data from the LIDAR sensor and determine an acceleration of the individual based on the first location, the second location, and additional locations based on the one or more additional sets of spatial data.

In another aspect, the present disclosure provides a method for monitoring an individual. The method includes receiving a first set of spatial data from a LIDAR sensor, calculating a first location of the individual relative to a support object based on the first set of spatial data, and determining if the first location is at an alert location relative to the support object. In some embodiments, calculating a first location of the individual relative to the support object may include distinguishing spatial data of the individual from spatial data of the support object, and calculating a center of mass of the individual based on the spatial data of the individual.

In some embodiments, determining an alert location of the individual relative to the support object includes calculating a location of at least one edge of the support object. In some embodiments, the alert location may be determined to be where the center of mass of the individual is beyond the edge of the support object. In some embodiments, the alert location may be determined to be where the center of mass of the individual is within a predetermined distance of the edge of the support object. In some embodiments, the method further includes sending an alert signal when the first location is determined to be at an alert location.

In some embodiments, the method further includes receiving a second set of spatial data from the LIDAR sensor, calculating a second location of the individual relative to the support object based on the second set of spatial data, and determining if the second location is at an alert location relative to the support object. The method may further include determining if the change from the first location to the second location is indicative of movement to an alert location. The method may further include calculating a probability that the individual will move to an alert location based on the first location and the second location. The method may further include determining a direction of movement of the individual. The method may further include determining a velocity of the individual.

In some embodiments, the method includes receiving one or more additional sets of spatial data from the LIDAR sensor and determining an acceleration of the individual based on the first location, the second location, and additional locations based on the one or more additional sets of spatial data. In some embodiments, the method further includes determining if the individual has moved from a recumbent position to a sitting position based on the first location and the second location.

### DESCRIPTION OF THE DRAWINGS

For a fuller understanding of the nature and objects of the disclosure, reference should be made to the following detailed description taken in conjunction with the accompanying drawings, in which:

FIG. 1 is a side elevation of a system according to an embodiment of the present disclosure, and including an individual on a bed;

FIG. 2A is a side elevation of a system according to an embodiment of the present disclosure with an individual in a supine position;

FIG. 2B is a cross-sectional view taken along A-A of FIG. 2A;

FIG. 2C is a cross-sectional view taken along B-B of FIG. 2A;

FIG. 2D is a cross-sectional view taken along C-C of FIG. 2A;

FIG. 2E is a cross-sectional view taken along D-D of FIG. 2A;

FIG. 3A is a diagram illustrating ranging data resulting from the cross-section of FIG. 2B;

FIG. 3B depicts the point cloud corresponding to FIG. 3A;

FIG. 4A is a side elevation of a system according to an embodiment of the present disclosure with an individual in a supine position and slightly inclined;

FIG. 4B is a cross-sectional view taken along E-E of FIG. 4A;

FIG. 4C is a cross-sectional view taken along F-F of FIG. 4A;

FIG. 4D is a cross-sectional view taken along G-G of FIG. 4A;

FIG. 4E is a cross-sectional view taken along H-H of FIG. 4A;

FIG. 5A is a side elevation of a system according to an embodiment of the present disclosure with an individual in a seated position;

FIG. 5B is a cross-sectional view taken along J-J of FIG. 5A;

FIG. 5C is a cross-sectional view taken along K-K of FIG. 5A;

FIG. 5D is a cross-sectional view taken along L-L of FIG. 5A;

FIG. 5E is a cross-sectional view taken along M-M of FIG. 5A;

FIG. 6A is a side elevation of a system according to an embodiment of the present disclosure with an individual in a seated position at the edge of the bed;

FIG. 6B is a cross-sectional view taken along N-N of FIG. 6A;

FIG. 6C is a cross-sectional view taken along P-P of FIG. 6A;

FIG. 6D is a cross-sectional view taken along Q-Q of FIG. 6A;

FIG. 6E is a cross-sectional view taken along R-R of FIG. 6A; and

FIG. 7 is a chart depicting a method according to another embodiment of the present disclosure.

### DETAILED DESCRIPTION OF THE DISCLOSURE

The present disclosure incorporates Light Detection and Ranging (LIDAR) technology to unobtrusively monitor an individual. A LIDAR sensor facilitates non-contact monitoring of patients on beds, chairs, and the like (support objects) and does not rely on detailed images of a patient to determine when a patient on a surface is attempting to exit the support surface. Embodiments of the present disclosure provide the ability to determine if an individual has left a support object (e.g., a supporting surface) such as a hospital bed, chair, etc. Furthermore, embodiments may provide the ability to predict that the individual is about to leave the support object, providing time for a caregiver to intercede and provide assistance to the individual.

The application of a LIDAR range sensing device to constantly monitor the surface of a patient support platform (chair, bed, etc), detecting the presence or absence of a patient, and monitoring the movement of a patient on a surface. The sensor signal can be processed to anticipate that the individual patient is attempting to exit from the support surface unexpectedly.

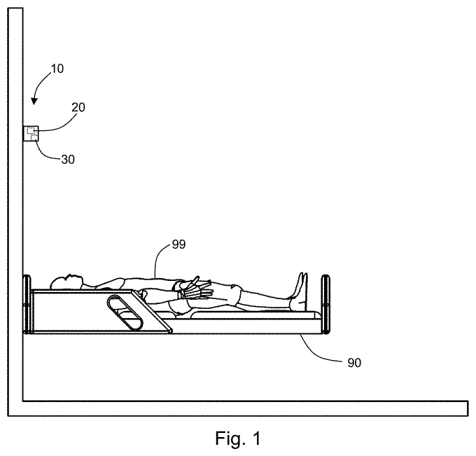

In an aspect, the present disclosure may be embodied as a system 10 for monitoring an individual (see, e.g., FIG. 1). The system 10 includes a processor 20 and a LIDAR sensor 30 in electronic communication with the processor 20. The LIDAR sensor 30 has a field of view in which it is capable of sensing ranges to one or more objects. It should be noted that the LIDAR sensor may be a 2-dimensional sensor in that the sensor is able to detect ranges to objects located in a plane (or in some cases, more than one plane) or a 3-dimensional sensor able to detect ranges to objects in a volume. To illustrate various embodiments, the present disclosure will be described using the non-limiting example of 2D LIDAR sensor monitoring a human individual supported on a bed.

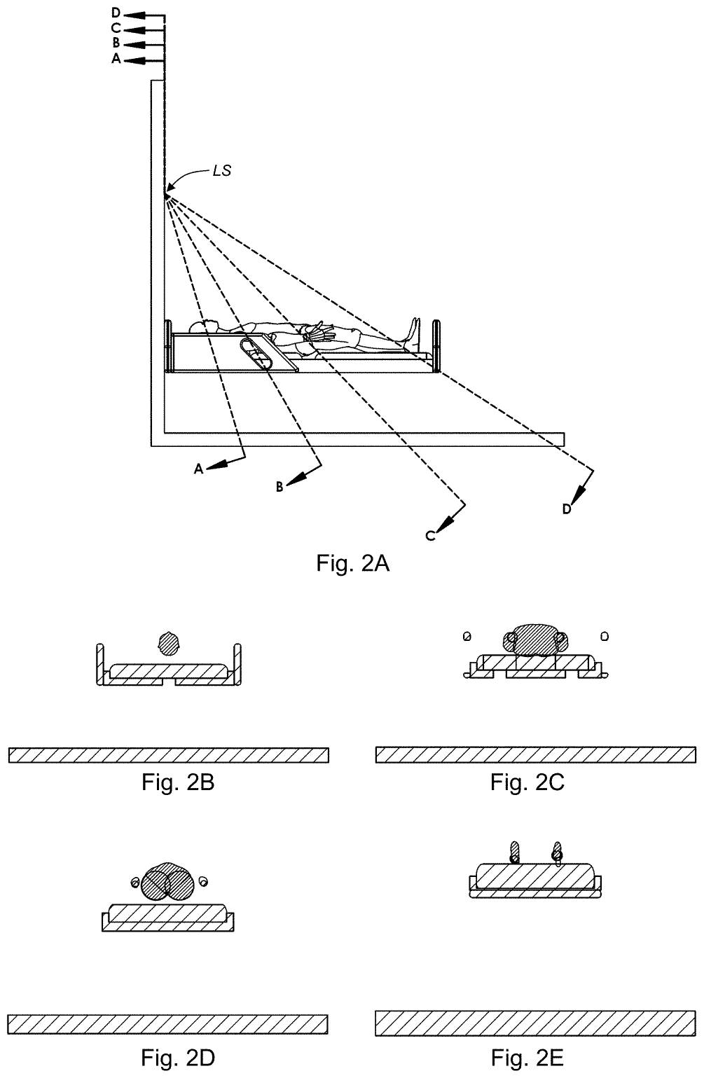

In a first scenario depicted in FIG. 2A, the individual 99 is lying supine on bed 90, and the LIDAR sensor (not shown, but located at the position labeled as ‘LS’) is configured such that the bed and the individual are within the field of view. The LIDAR sensor may be configured for range finding in one or more pre-determined planes. For example, the LIDAR sensor may be configured to find the range to objects within one or more of the planes indicated with the sections lines A-A, B-B, C-C, and/or D-D. When configured on plane A-A, the LIDAR sensor will find the range to the individual's head and the bed proximate the head (see FIG. 2B). On plane B-B, the LIDAR sensor will find the range to the individual's chest and the bed proximate the chest (see FIG. 2C). On plane C-C, the LIDAR sensor will find the range to the individual's pelvic area and the bed proximate the pelvis (see FIG. 2D). On plane D-D, the LIDAR sensor will find the range to the individual's lower legs and the bed proximate the lower legs (see FIG. 2E).

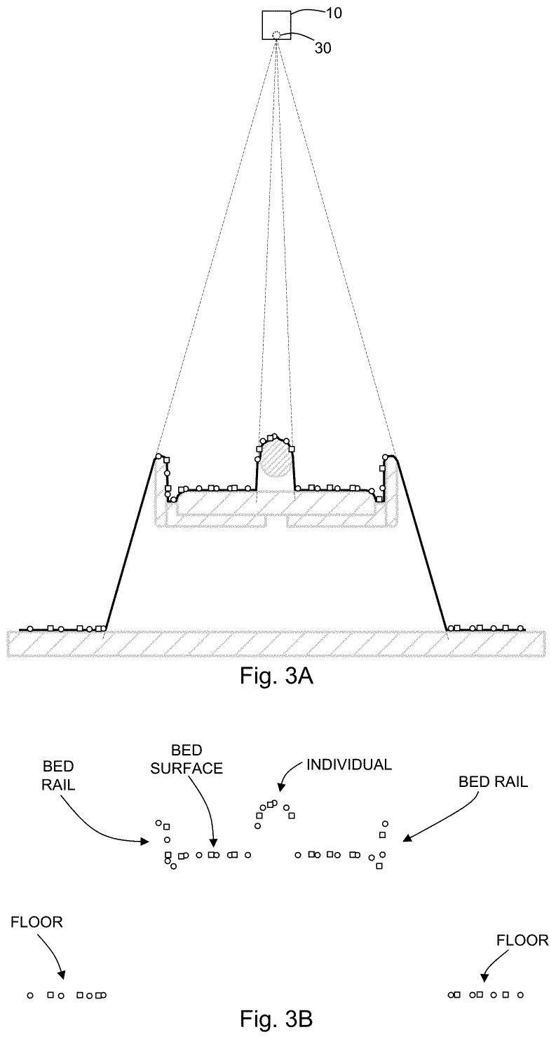

Although FIGS. 2B-2E depict complete cross-sections on each of the corresponding planes, it will be apparent to one familiar with LIDAR technology that the LIDAR sensor will provide a range only to the first object detected at any sampled position on its scanning plane. FIGS. 3A and 3B illustrate this concept with respect to the cross-section of FIG. 2B (corresponding to A-A of FIG. 2A). As the LIDAR sensor scans across its scanning plane (in this case aligned with A-A), the sensor will sample a range to an object at each sampling point (indicated by small circles and squares). Each of these points will fall on the solid black line. The LIDAR sensor will repeatedly scan along its scanning plane such that, for example, one a first pass of the scanning plane, the spatial data includes the range at each point indicated by the small circles, and on a second pass of the scanning plane, the spatial data includes the range at each point indicated by the small squares. The resulting spatial data is a point cloud such as that depicted in FIG. 3B. The LIDAR sensor may be configured to limit the range of the spatial data such that, for example, the floor of the room is not measured or the measurements are ignored.

The processor 20 is configured to receive a set of spatial data from the LIDAR sensor 30. The received spatial data may be used to calculate a first location of the individual. A priori information is used to determine a support surface of the support object, other fixed features of the support object (such as, for example, bed rails), and other room features (such as, for example, the floor, nightstand, etc.) In this way, the spatial data of the individual may be distinguished from the spatial data of the support object. In some embodiments, a center of mass of the individual is calculated based on the spatial data of the individual. In this way, the first location may be the location of the center of mass of the individual. The first location may be calculated with relative to the support object. The processor may also be configured to determine if the first location is at an alert location relative to the support object. For example, the alert location may be at or near an edge of the support object (e.g., an edge of the bed). In some embodiments, the alert location may be beyond the edge of the support object. In such an example, the processor may be configured to determine if the individual has left the bed (whether intentional or not). In some embodiments, the alert location is determined if the center of mass of the individual is within a predetermined distance of the edge of the support object.

The processor 20 may be configured to send an alert signal when the first location is determined to be at the alert location. For example, the alert signal may be an audible alarm, a visual alarm, a haptic alarm, an electronic signal to a separate device (e.g., a signal to a nurse call system, a text message to a smart phone, etc.), or any other type of alert or combination of such alerts.

In some embodiments, the process 20 may be configured to receive a second set of spatial data from the LIDAR sensor 30. For example, as described above, the LIDAR sensor may scan its field of view over multiple passes, and a second set of spatial data may correspond to a second pass of the sensor. In other embodiments, the each of the first set and second set of spatial data may be made up of multiple passes of the LIDAR sensor. A second location of the individual is calculated based on the second set of spatial data. The second location may be calculated relative to the support object. The processor may determine if the second location is at an alert location relative to the support object.

In some embodiments, the processor 20 is further configured to determine if the change from the first location to the second location is indicative of movement to an alert location. For example, the processor may determine a direction of travel of the individual and/or a velocity of the individual. The processor may calculate a probability that the individual will move to the alert location. The calculation of probability may be based on the first location and the second location. For example, the calculation of probability may be based on the velocity and/or direction of the individual. In this way, if the location of the individual (e.g., the center of mass of the individual) is determined to be moving quickly in the direction of the edge of the bed, the probability of the individual moving to the edge of the bed may be high-indicating the individual is leaving the bed.

The scanning plane may be pre-determined. For example, the scanning plane may be configured (e.g., the LIDAR sensor positioned) based on knowledge of the support object configuration. With reference to FIG. 2A, the scanning plane corresponding to sections lines C-C may be more relevant that the planes along A-A and B-B due to the presence of the bed rail (thereby preventing the individual from exiting the bed at those positions. Similarly, the plane of C-C may be more relevant than the plane of D-D because some an individual's feet or lower legs may move beyond the edge of the bed without necessarily indicating that the individual is off of the bed (or moving off of the bed). In some embodiments, the LIDAR sensor may be configured to scan along more than one plane. For example, with reference to FIG. 2A, the LIDAR sensor may be configured to scan on more than one of the planes generally indicated by section lines A-A, B-B, C-C, and D-D. Such a configuration may be useful where, for example, the individual is free to lower their bed rails, has a movable table positioned at a location over the bed, etc. In this way, the processor may be configured to alert based on information in one or more of the planes and/or disregard information in other planes.

In some embodiments, the processor 20 is further configured to receive additional sets of spatial data from the LIDAR sensor 30 (e.g., a third set, a fourth set, etc.) Such additional sets of spatial data may be used to parameters such as updated velocity(ies), acceleration, and/or updated location(s), etc.

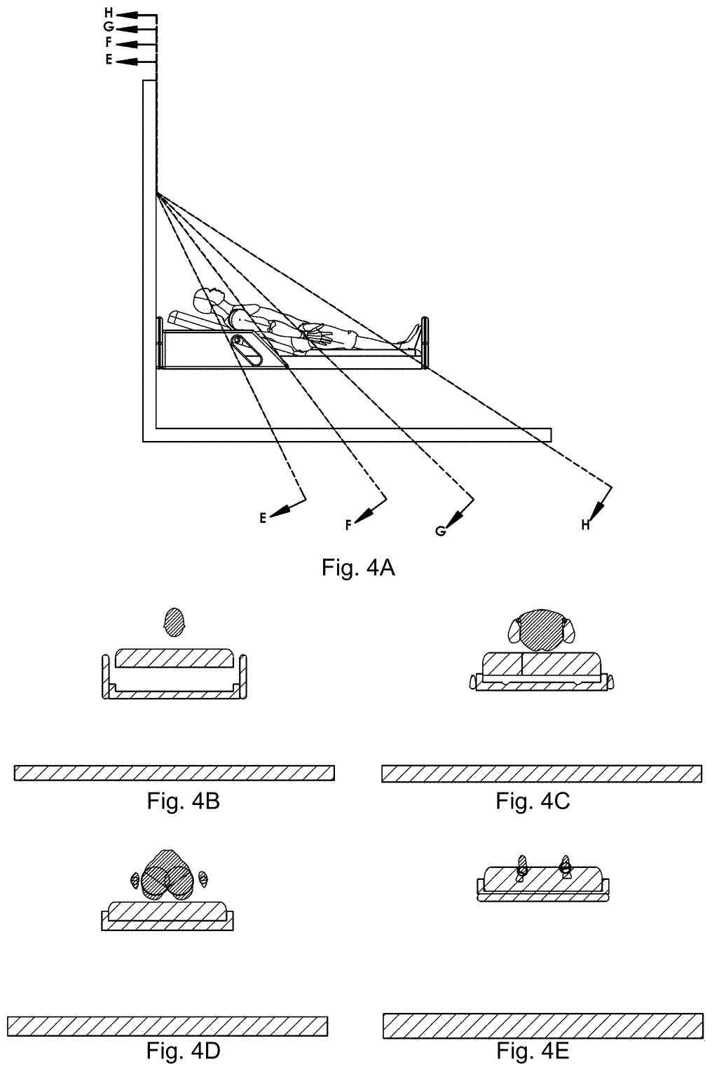

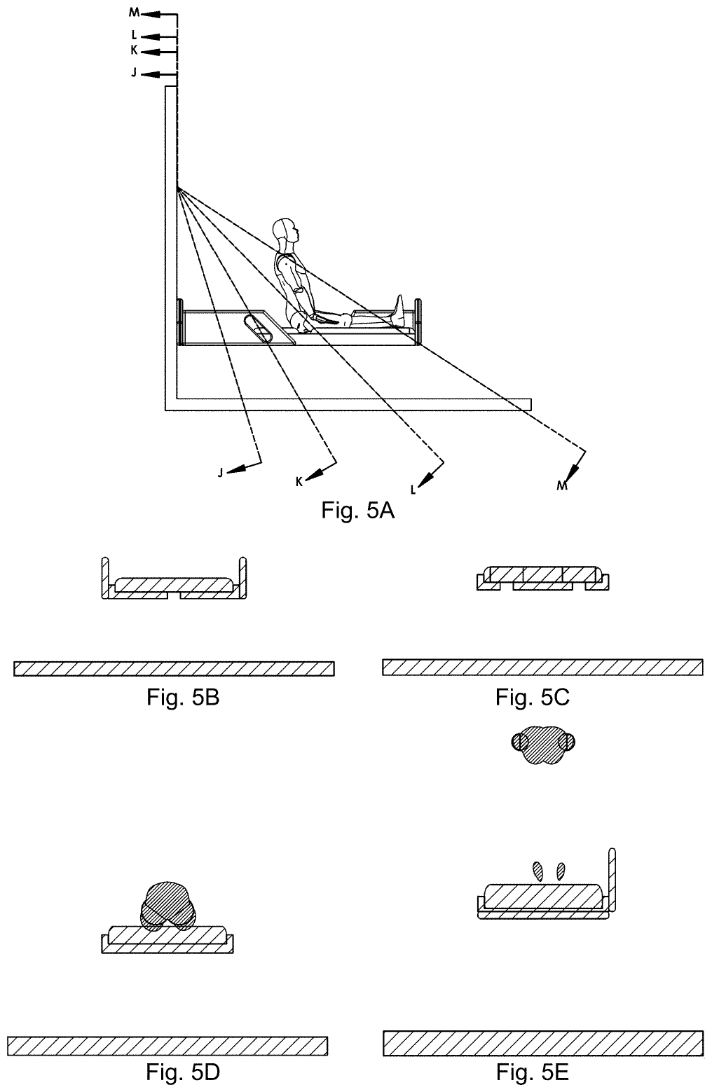

In some embodiments, the processor may determine if the individual has moved from a reclined position to a less reclined position. For example, the individual may raise the head of the bed such that they are less reclined. FIG. 4A depicts such a scenario where the individual has raised the head of the bed (changed the inclination of the bed). It is noted that the vertical location of the individual (e.g., the vertical location of the individual's center of mass) is fairly constant with respect to the bed surface (see FIGS. 4B-4E). In another example, the individual may sit up in bed. For example, FIG. 5A depicts where the individual is now in a sitting position (though still remaining in bed). In such a scenario, the vertical location of the individual has changed relative to the bed surface. For example, FIG. 5E shows that the individual's vertical location is substantially higher than the bed surface. It is noted that FIGS. 5B and 5C do not include the individual at all because of the individual's seated position.

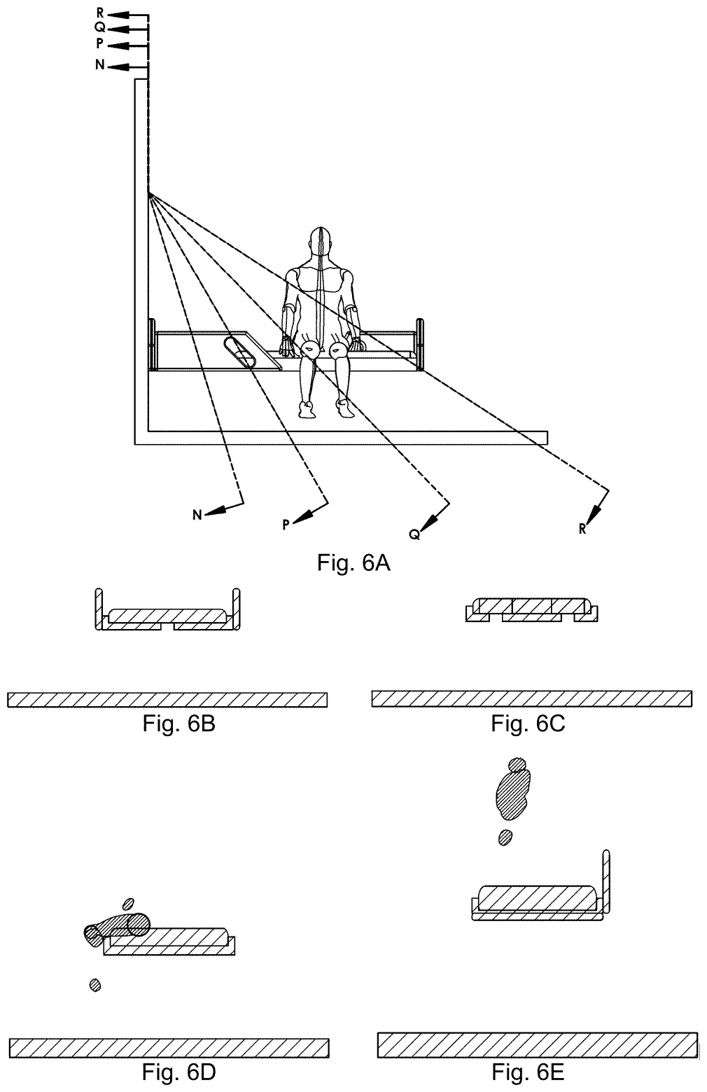

FIGS. 6A-6E depict a scenario where the individual is in a seated position and at the edge of the bed, such that the legs are over the edge of the bed. It can be seen that the individual is not present in FIGS. 6B and 6C (planes N-N and P-P). FIG. 6C (Q-Q) shows the individuals wrist, upper leg and kneed. The center of mass in FIG. 6C is very near the edge of the bed. FIG. 6E shows the individual's upper arm and torso and indicates horizontal movement of the center of mass as compared to the spatial data of FIG. 5E. In this way, movement of the center of mass from the location in FIG. 4E to that of FIG. 5E would indicate that they individual has moved to a sitting position, and movement from the location in FIG. 5E to the location of FIG. 6E indicates a movement towards the edge of the bed. Such a movement may indicate that the individual is about to leave the bed. In such a scenario, the processor may calculate a high probability that the individual will move to an alert location (e.g., beyond the edge of the bed).

A LIDAR device provides distance and angular position measurements from a fixed point which, when communicated to a computing device, can be used to create a polar coordinate or Cartesian coordinate model of an environment, including objects in the environment. A LIDAR device typically provides individual measurement data such as distance and angle pairs in rapid succession in time, which are typically illustrated as points. Collections of this type of data are usually graphed, generating visible points in such close proximity to each other that a human-viewable image of the data is possible.

In this illustration, a 2 dimensional LIDAR (360° line scan) device mounted in a fixed position (near the top of a room) is used to generate a model of a room environment. The LIDAR device is positioned so as to include a scan across a patient surface, typically aimed to include an area of the surface most likely to be occupied by a patient when the patient is using the patient surface. In this image, the patient support surface can be defined by two edges, and is observed to most likely be empty (no significant disturbance on the patient surface).

To more accurately detect a movement of a patient from a centered position to an edge position, the LIDAR data can be used to estimate changes in the center of mass or center of gravity of the patient. Several techniques are suitable for this purpose. For example, the center of mass calculation may be based on the summation of the products of individual distance measurement element height (distance from the support surface to measured individual height) and the distance element width (distance between measured points, correlated to the measurement angle) across the individual combined with an assumption of homogeneous patient composition (density) can be used to approximate an instantaneous center of mass. This may be compared with subsequent center of mass calculations to determine when a pattern of movement of the center of mass of the patient toward an edge of the support surface is an attempt by the individual to exit the surface. When movement of the individual's center of mass toward an edge is detected, a notification can be sent to caregiver(s) indicating that the individual may be attempting to exit the bed.

A second technique for using LIDAR sensing to predict or anticipate a user attempting to exit from a bed or other patient surface is a method where a virtual plane is established by the LIDAR sensor over or around a patient area, and unexpected changes or interruptions in the virtual plane provides an indication of patient activity. The virtual plane can be established above a patient seated on a chair or seating surface. As the patient attempts to stand, portions of their body interrupt the plane and are detected, resulting in a notification being sent to caregiver(s) indicating the activity. In another embodiment, a virtual plane is established over the bed of a patient, and activity such as sitting up in the bed projects a portion of the body of the patient through the plane, resulting in a break or change in the virtual plane that is detected and converted to a notification sent to caregiver(s).

The system may be configured in different ways with respect to the support object, room configuration, etc. For example, in some embodiments, the system may be affixed to a wall of the room. Such a configuration is advantageous because the system may also be used to detect the presence of a bed. In this way, if no bed is present, the system may disable any alerts (e.g., false alerts), provide information to other systems indicating no patient is located in the room, etc. Furthermore, such a configuration allows rooms to be reconfigured without the need to reconfigure the system (e.g., associate the LIDAR system with a new room, etc.) In other embodiments, the system may be affixed to the support object thereby providing advantages such as a potentially-improved knowledge of the support object configuration for more accurate calculations.

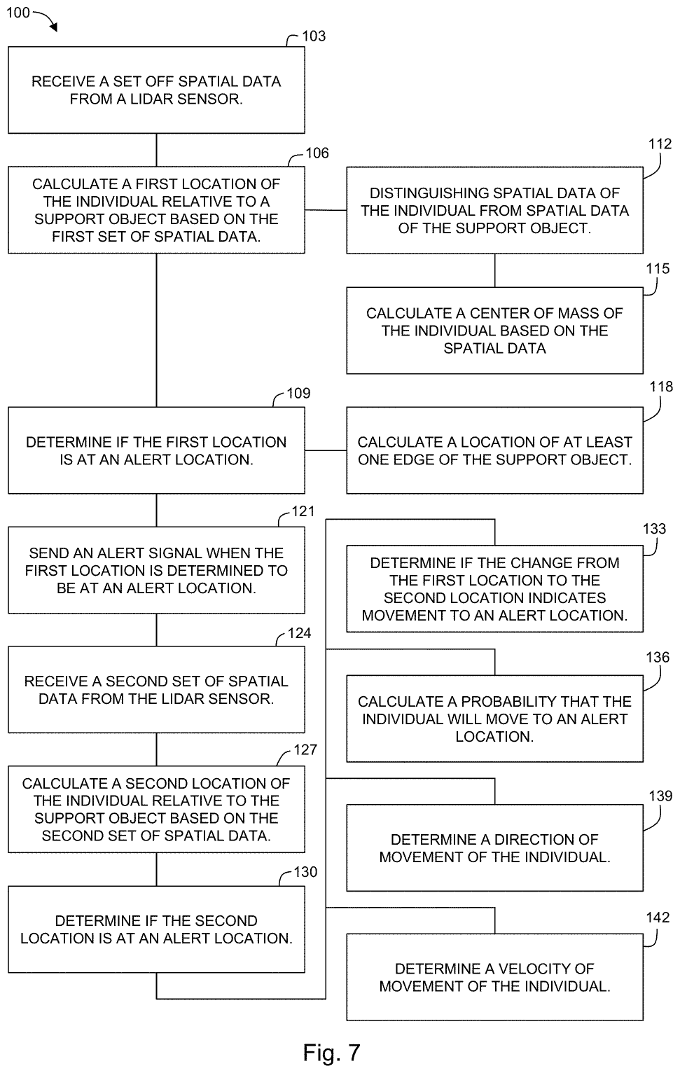

With reference to FIG. 7, in another aspect, the present disclosure may be embodied as a method 100 for monitoring an individual. The method 100 includes receiving 103 a first set of spatial data from a LIDAR sensor. A first location of the individual is calculated 106. The first location may be calculated relative to a support object based on the received 103 first set of spatial data. The method 100 includes determining 109 if (i.e., whether, when, etc.) the first location is at an alert location relative to the support object. The first location may be calculated by distinguishing 112 spatial data of the individual (i.e., within the first set of spatial data) from spatial data of the support object. A center of mass of the individual may be calculated 115 based on the distinguished 112 spatial data of the individual.

In some embodiments, determining 109 an alert location of the individual relative to the support object includes calculating 118 a location of at least one edge of the support object. In some embodiments, the alert location may be determined if the center of mass of the individual is beyond the edge of the support object (i.e., the individual is determined to be at the alert location if the center of mass of the individual is beyond the edge of the support object). In some embodiments, the alert location may be determined if the center of mass of the individual is within a predetermined distance of the edge of the support object. The method 100 may include sending 121 an alert signal when the first location is determined to be at an alert location.

In some embodiments, the method 100 include receiving 124 a second set of spatial data from the LIDAR sensor. A second location of the individual is calculated 127 relative to the support object based on the second set of spatial data. The method may include determining 130 if the second location is at an alert location relative to the support object. The method 100 may include determining 133 if the change from the first location to the second location is indicative of movement to an alert location. The method 100 may include calculating 136 a probability that the individual will move to an alert location based on the first location and/or the second location. The method 100 may include determining 139 a direction of movement of the individual. The method 100 may include determining 142 a velocity of the individual. The movement and/or velocity may be determined using the first location and the second location (for example, using the locations of the center of mass of the individual).

In some embodiments, the method may further comprise receiving one or more additional sets of spatial data from the LIDAR sensor. Such one or more additional sets of spatial data may be used with the first location and/or the second location to determine additional characteristics of the individual. For example, an acceleration of the individual may be determined using the one or more additional sets of spatial data (e.g., one or more additional locations of the individual) either alone or in combination with the first location and/or the second location.

In some embodiments, the method include determining if the individual has moved from a recumbent position to a sitting position (seated position) based on one or more of the first location, the second location, and the one or more additional locations.

The systems and methods of this disclosure are described for convenience with 2-dimensional or single-plane LIDAR devices. Such 2D devices may provide an economical deployment of the technology. The same concepts apply to, and may benefit from, the deployment of 3-dimensional LIDAR devices, which is within scope of the present disclosure. The use of 3-dimensional LIDAR would make multiple planes or surfaces available for use in detecting patient activity. Additionally, the use of LIDAR for the purposes shared in this disclosure also benefit from the use of LIDAR devices that have limited or narrowed ranging areas, reducing or eliminating the need to sort through additional data provided by LIDAR units which may sweep or scan a full 360° range when in use.

Although the present disclosure has been described with respect to one or more particular embodiments, it will be understood that other embodiments of the present disclosure may be made without departing from the spirit and scope of the present disclosure.

We claim:

1. A system for monitoring an individual, comprising:
a processor;
a LIDAR sensor in electronic communication with the processor; and
wherein the processor is configured to:
receive a set of spatial data from the LIDAR sensor;
calculate a first location of the individual relative to a support object based on the set of spatial data; and
determine if the first location is at an alert location relative to the support object.

2. The system of claim 1, wherein the processor is configured to calculate the first location of the individual relative to the support object by:
distinguishing spatial data of the individual from spatial data of the support object; and
calculating a center of mass of the individual based on the spatial data of the individual.

3. The system of claim 1, where the processor is configured to determine an alert position of the individual relative to the support object by calculating a location of at least one edge of the support object.

4. The system of claim 3, wherein the alert location is determined if the center of mass of the individual is beyond the edge of the support object.

5. The system of claim 3, wherein the alert location is determined if the center of mass of the individual is within a predetermined distance of the edge of the support object.

6. The system of claim 1, wherein the processor is configured to send an alert signal when the first location is determined to be at the alert location.

7. The system of claim 1, wherein the processor is configured to:
receive a second set of spatial data from the LIDAR sensor;
calculate a second location of the individual relative to the support object based on the second set of spatial data; and
determine if the second location is at an alert location relative to the support object.

8. The system of claim 7, wherein the processor is configured to:
determine if the change from the first location to the second location is indicative of movement to an alert location.

9. The system of claim 8, wherein the processor is configured to:
calculate a probability that the individual will move to an alert location based on the first location and the second location.

10. The system of claim 7, wherein the processor is configured to:
determine a direction of movement of the individual.

11. The system of claim 7, wherein the processor is configured to:
determine a velocity of the individual.

12. The system of claim 7, wherein the processor is configured to:
receive one or more additional sets of spatial data from the LIDAR sensor; and
determine an acceleration of the individual based on the first location, the second location, and additional locations based on the one or more additional sets of spatial data.

13. The system of claim 7, wherein the processor is configured to:
determine if the individual has moved from a recumbent position to a sitting position based on the first location and the second location.

14. A method for monitoring an individual, comprising:
receiving a first set of spatial data from a LIDAR sensor;
calculating a first location of the individual relative to a support object based on the first set of spatial data; and
determining if the first location is at an alert location relative to the support object.

15. The method of claim 14, wherein calculating a first location of the individual relative to the support object further comprises:
distinguishing spatial data of the individual from spatial data of the support object; and
calculating a center of mass of the individual based on the spatial data of the individual.

16. The method of claim 14, where determining an alert location of the individual relative to the support object further comprises calculating a location of at least one edge of the support object.

17. The method of claim 16, wherein the alert location is determined if the center of mass of the individual is beyond the edge of the support object.

18. The method of claim 16, wherein the alert location is determined if the center of mass of the individual is within a predetermined distance of the edge of the support object.

19. The method of claim 14, further comprising sending an alert signal when the first location is determined to be at an alert location.

20. The method of claim 15, further comprising:
receiving a second set of spatial data from the LIDAR sensor;
calculating a second location of the individual relative to the support object based on the second set of spatial data; and
determining if the second location is at an alert location relative to the support object.

21. The method of claim 20, further comprising determining if the change from the first location to the second location is indicative of movement to an alert location.

22. The method of claim 21, further comprising calculating a probability that the individual will move to an alert location based on the first location and the second location.

23. The method of claim 20, further comprising determining a direction of movement of the individual.

24. The method of claim 20, further comprising determining a velocity of the individual.

25. The method of claim 20, further comprising:
receiving one or more additional sets of spatial data from the LIDAR sensor; and
determining an acceleration of the individual based on the first location, the second location, and additional locations based on the one or more additional sets of spatial data.

26. The method of claim 20, further comprising:
determining if the individual has moved from a recumbent position to a sitting position based on the first location and the second location.
